(19) United States
(12) Patent Application Publication
(10) Pub. No.: US 2003/0143136 A1
(43) Pub. Date: Jul. 31, 2003

(54) Instrument sterilization roll

(76) Inventor: Timothy P. Regan, Archbald, PA (US)

Correspondence Address: Timothy P. Regan, 129 Maria Blvd., Archbald, PA, 18403, US

(21) Appl. No.: 09/901,388

(22) Filed: Jan. 25, 2002

(51) Int. Cl. A61L 9/00

(52) U.S. Cl. 422/294

(57) ABSTRACT

The Instrument Sterilization-Roll is innovative because it uses existing materials in a new design to allow for an efficient, inexpensive and time conserving method of sterilization. The new design incorporates a perforation between pouches, which can be of any functional width, connecting them allowing separation while maintaining sterility. Once the instruments are processed, the row of instruments can be rolled to facilitate ease of storage. The counting aid allows quick counting of the number of pouches removed from the roll at any given time. The fact that the pouches are connected allows the sealing of numerous pouches at once when the protective strip is removed revealing an adhesive strip. The flap is folded sealing the whole row of pouches at once. With the individual pouches each strip needs to be removed and folded. This is very time consuming. The existing pouches are large allowing numerous or large instruments to be sterilized. When sterilizing numerous instruments or small instruments with the old pouches there are serious drawbacks. If sterilizing single instruments, the present systems become expensive and time consuming. If numerous instruments are sterilized under the present systems and only one or a few are needed the whole pack of instruments must be resterilized again. The Instrument Sterilization Roll combines visualization, efficiency, sterilization maintenance, and cost effectiveness, making it practical to sterilize every instrument and maintain that sterilization until the time of use.

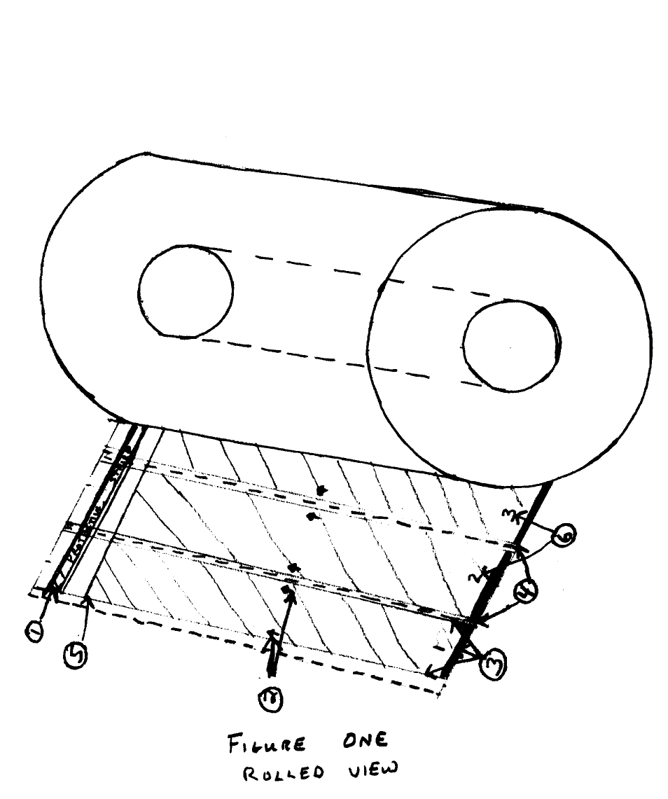

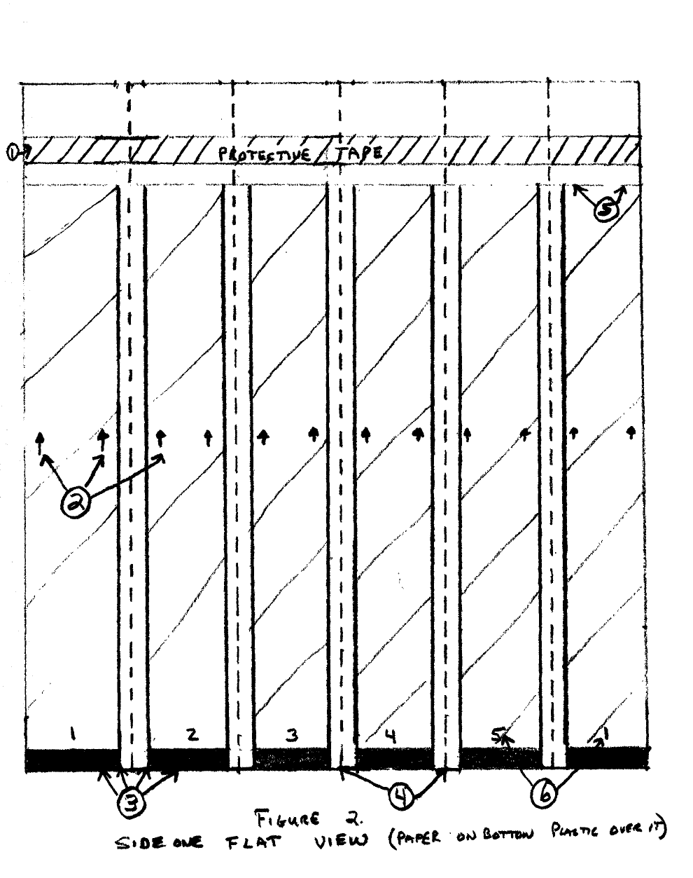

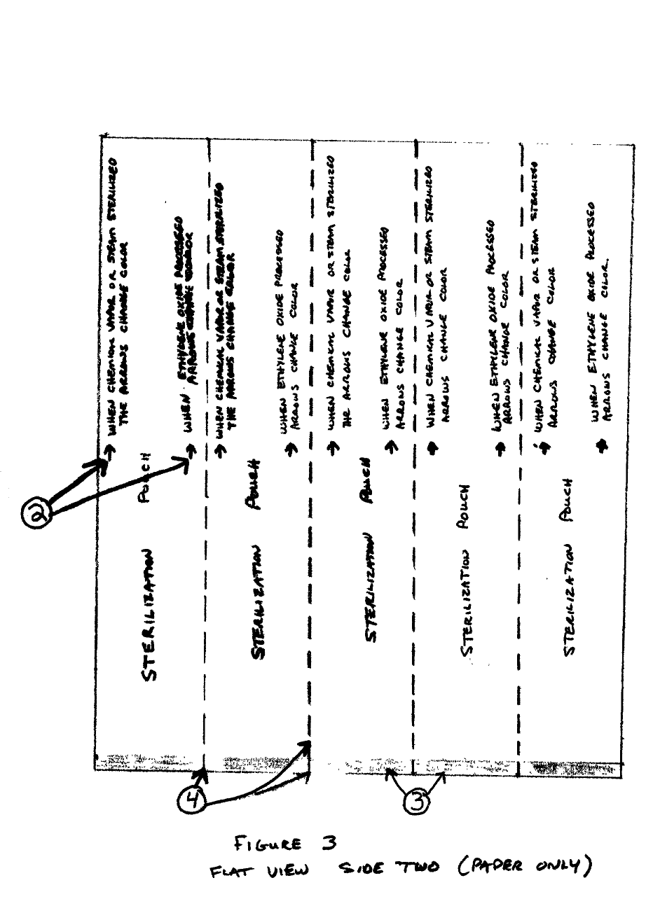

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO A MICROFICHE APPENDIX

Not Applicable

### BACKGROUND OF THE INVENTION

The invention pertains to the materials used to sterilize dental or medical hand instruments. Previous designs caused sterilization of single instruments to bee costly, time consuming, and difficult to store. The new design allows efficient sterilization, maintenance of sterility of each instrument until the seal of each pouch is compromised, and the storage of the instruments easier making more efficient use of space.

### BRIEF SUMMARY OF THE INVENTION

The Instrument Sterilization Roll is a series of sterilization pouches large enough for a single instrument. The series of pouches are connected in one roll by perforations allowing separation at any point along the strip. Each pouch has two layers, a paper side, which is covered with plastic except for the top third. These two layers are sealed on both sides and at the bottom. This creates a pouch which when the top third of the paper layer is folded over creates a closed pouch. The top third has a protective strip covering an adhesive strip that keeps the seal when the top flap is folded onto the plastic layer. This allows the instrument to be sealed into the pouch. This maintains sterility until the pouch is opened. Instruments can be placed in any order and once sterilized can be rolled to facilitate ease of storage.

### BRIEF DESCRIPTION OF THE SEVERAL VIEWS OF THE DRAWINGS

FIG. One: FIG. one depicts the rolled view of the product. In this way the pouches can be dispensed easily. The entire roll can be placed on a towel rack allowing for easy access to the pouches. The legend is as follows:

Number (1): Protective Strip: Shows the protective strip that covers an adhesive strip underneath which when folded creates a seal between the plastic and paper edges.

Number (2): Sterilization Verification Arrows: These arrows will verify whether the sterilization process has occurred. These arrows will change color when sterilized with either chemical vapor; steam or ethylene oxide is used.

Number (3): Sealed Edges: These edges represent the union of the paper and plastic edges on both sides and at the bottom. This creates a pouch, open at the top.

Number (4): Perforation Lines: These perforations allow the separation of pouches at any time.

Number (5): Open Edge: This is the only edge not factory sealed. This allows the separation of the paper and plastic layers.

Number (6): Counting Aid: These are the numbers placed 1 through 5, placed continuously on the roll to aid in the counting of the number of pouches needed at any given time.

FIG. Two: This is the flat view. The paper is facing down with the plastic layer on top. The legend is the same as above using the same numbers seen on the rolled view.

FIG. Three: This is the flat view with the paper side up. In this view the plastic layer is hidden. The paper side extends longer than the plastic layer so the fold can be done to create a seal. There are two sterilization conformation arrows. One of the arrows changes color when a chemical vapor or steam sterilization process is used. The other arrow changes color when the Ethylene Oxide process is used.

### DETAILED DESCRIPTION OF THE INVENTION

The Instrument Sterilization Roll is a product that is to be used for the purpose of maintaining sterilization of dental or medical instruments. The product consists of two layers. The bottom layer is made of heat and chemiclave resistant paper, which will withstand the sterilization process of heat or chemical autoclaves. The second layer or top layer is made of a translucent plastic that is also able to withstand both heat and chemical sterilization. These materials are currently used routinely in sterilization pouches made for dental and medical instruments.

Layer One (the paper side) will have a protective strip of tape which when removed will uncover an adhesive strip that will be used to seal the pouch when folded to overlap layer two (transparent plastic side). This seal will maintain sterility within the pouch after sterilization is achieved. On the backside of the paper opposite where the plastic side is, there are sterilization conformation arrows. One of these arrows will be sensitive to chemical vapor and steam sterilization and will change color when the pouch is subjected to these types of sterilization. The other arrow will be sensitive to ethylene oxide sterilization and will change color when subject to that sterilization modality. At the bottom of each pouch will be a counting aid. The pouches will be numbered from one to five in series, continuously throughout the length of the roll of pouches. This will allow for easy counting of the number of pouches removed from the roll.

Layer Two is a transparent plastic layer. This plastic will be made of a material that can withstand the various forms of sterilization used. Both sides as well as the bottom edges of the plastic will be sealed to the underlying paper layer. When the paper layer is folded over onto the plastic layer, after removing the protective strip, the adhesive on the paper will create a seal. This seal will maintain the sterility of the enclosed instrument.

The pouches will be supplied on one continuous roll with each pouch separated by a perforation. This allows the separation of any desired number of pouches needed at any given time. After the instruments in each pouch in the row, the protective tape can be removed from the whole row and sealed in on quick motion sealing the instruments and making them ready for the sterilization process. Once the sterilization cycle is complete, the pouches can be removed and stored as a strip or a roll until needed.

The improvements over the old pouches include the perforations, which allow separation of instruments within a group, limiting the number of instruments sterilized at any given time. Also, the counting aid makes it easy to determine how many pouches are separated from the roll at any given time. The fact that the pouches are dispensed on a roll makes it much easier to store and creates easier access to the pouches. Previously the pouches came in boxes of individual pouches. After each instrument is placed into the pouch the tape is removed and the seal is created. This method is very time consuming when numerous small instruments are being sterilized. Also, when a series of instruments needed for the same procedure are sterilized instead of being placed into the same pouch, they can be sterilized in a series creating an instrument pack. If one of the necessary instruments is missing and the same pouch system is used, another pack will have to be opened exposing instruments not needed requiring all of the instruments to be processed, repacked, and sterilized. If the instrument sterilization roll is used only the instruments needed will be exposed and then sterilized.

1. I claim that my invention, The Instrument Sterilization Roll, is a redesign of the sterilization pouch used to maintain sterilization of medical or dental instruments. Instrument sterilization is an extremely important step in the prevention of cross contamination caused by non-disposable instruments used in dentistry and medicine: There have been systems created to attempt to achieve this goal However, most systems are either bulky, inefficient, or have serious drawbacks. If the existing systems allow sterilization of numerous instruments at one time the problem is that once the integrity of the system is broken all of the instruments, used or unused, have to be reprocessed. This creates the need for unnecessary repetition to create the sterile instrument. The Instrument Sterilization Roll allows for the sterilization of each individual instrument while creating groups that may be desirable for different procedures, while saving time, effort and materials.
